(19) United States
(12) Patent Application Publication
(10) Pub. No.: US 2004/0220264 A1
(43) Pub. Date: Nov. 4, 2004

(54) Bioavailability and improved delivery of acidic pharmaceutical drugs

(76) Inventors: Ruey J. Yu, Chalfont, PA (US); Eugene J. Van Scott, Abington, PA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/801,134

(22) Filed: Mar. 16, 2004

Related U.S. Application Data
(60) Provisional application No. 60/454,631, filed on Mar. 17, 2003.

(51) Int. Cl. A61K 31/205

(52) U.S. Cl. 514/554

(57) ABSTRACT

Embodiments of the invention relate to a composition, a process of making the composition, and to the use of the composition. The compositions include a molecular complex formed between an acidic pharmaceutical drug and at least one functional substance. The compositions provide improved bioavailability and improved delivery of the drug into the cutaneous tissues.

### BACKGROUND OF THE INVENTION

1. Field of the Invention

Embodiments of the invention relate to a process of making and the use of topical compositions including a molecular complex formed between an acidic pharmaceutical drug and at least one functional substance. The compositions provide improved bioavailability and improved delivery of the drug into the cutaneous tissues. The molecular complex thus formed is bioavailable for cutaneous penetration and is less or non-irritating for topical management or treatment of dermatological disorders and other cutaneous indications.

2. Description of Related Art

Transdermal delivery systems are a convenient and effective alternative for the administration of many types of medications, because the agents are delivered directly into the blood stream, avoiding first-pass metabolism in the liver, so that drug delivery is continuous and sustained. Transdermal delivery also provides a sustained and consistent delivery of medication, avoiding peaks and valleys in blood levels which are often associated with oral dosage forms. Thus, using transdermal delivery, one can administer lower doses of drug to achieve the same therapeutic effect compared to oral administration, reducing or eliminating dose-dependent side effects.

Preparing suitable formulations of medications is a challenging task. The skin, which has protective layers designed to prevent penetration of foreign matter, must be sufficiently penetrated to provide the active agent to the desired site or for absorption into the bloodstream. Skin is a complex organ system, consisting of multiple layers. The uppermost, or “stratum corneum,” layer of skin consists of non-living material derived primarily from the terminal differentiation of epidermal keratinocytes, and provides a protective barrier for the underlying components of skin. The epidermis contains a number of cell types, although keratinocytes are the major cell type. Dermal fibroblasts are embedded within a matrix comprised of collagen, elastin, proteoglycans, and other extracellular matrix molecules. Blood capillaries are found in the dermis, but the epidermis is non-vascular.

In addition, the drug itself must be suitable for administration. The size of a drug molecule, its charge, polarity, and pH are factors that contribute to the ability of the agent to penetrate the skin to the desired site or to blood vessels for systemic distribution. The carrier enabling the transdermal delivery of the drug has similar constraints.

Most transdermal delivery of pharmaceuticals involves incorporating the pharmaceutical into a carrier, such as a porous polymeric membrane, and using the membrane as a patch worn on the skin. Transdermal patch devices which provide a controlled, continuous administration of a therapeutic agent through the skin are known as the art. Such devices, for example, are disclosed in U.S. Pat. Nos. 4,627,429; 4,784,857; 5,662,925; 5,788,983; and 6,113,940. These devices typically contain a therapeutic agent impermeable barrier layer that defines the outer surface of the device, and a permeable skin attaching membrane, such as an adhesive layer, sealed to the barrier layer in such a way as to create a reservoir between them in which the therapeutic agent is placed. Although such devices may be satisfactory for their intended purpose, they have been found to be irritating to the wearer of the patch, provide minimized control of drug delivery through the skin, are slower to prepare, do not allow for customized formulation, are not easily produced, and are not cost-effective.

Numerous chemical agents have been studied as a means of increasing the rate at which a drug penetrates through the skin. As will be appreciated by those skilled in the art, chemical enhancers are compounds that are administered along with the drug (or in some cases the skin may be pretreated with a chemical enhancer) in order to increase the permeability of the stratum corneum, and thereby provide for enhanced penetration of the drug through the skin. Ideally, such chemical penetration enhancers are compounds that are innocuous and serve merely to facilitate diffusion of the drug through the stratum corneum. The permeability of many therapeutic agents with diverse physicochemical characteristics may be enhanced using these chemical enhancement means. However, there are skin irritation and sensitization problems associated with high levels of certain enhancers.

Some medicinal active agents contain one or more acidic groups in their molecule and can therefore be utilized in pharmaceutical preparations either as a free acid or as a salt of the active substance acid with an alkali which is suitable for this purpose. Salts have the advantage of better water solubility, which is important for oral administration, and in many cases also the advantage of better stability. A further advantage is that active substance salts often are more easily crystallized, or it is anyway only the active substance salt which is crystalline at room temperature. This is the reason why many active substances are manufactured and available only in the form of their salts. For example, ibuprofen and theophylline are commercially available for oral administration as ibuprofen lysine salt, ibuprofen methylglucamine salt and theophylline aminoisobutanol salt.

For transdermal administration, however, the active substance salts are unsuitable. Due to their higher polarity and presence as a negatively charged anion, they are not capable of penetrating the lipophile barrier of the stratum corneum in the quantities required for the therapeutic purpose. Thus, it is necessary to transform active substance salts into their free acid in order to utilize them in transdermal systems. Processes of making a topical composition comprising molecular complexes of these drugs with other vehicles for optimal bioavailability and improved delivery into the cutaneous tissues has not previously been described.

U.S. Pat. No. 5,877,212, the disclosure of which is incorporated by reference herein in its entirety, discloses molecular complexes and sustained release formulations containing complexes formed between alpha hydroxyacids and related acids on the one hand, and a complexing agent on the other hand. The complexing agents include organic amino compounds in free base form having one or more other functional groups with unshared electrons such as hydroxyl, carbonyl, amido, ester, and alkoxy groups. The molecular complex provides for controlled release of the alpha hydroxyacid or related acid into the skin.

The description herein of certain disadvantages of known materials, methods, systems, and apparatus is not intended to limit the scope of the invention. Indeed, various embodiments of the invention may include some or all of the known materials, methods, systems, and apparatus without suffering from the aforementioned disadvantages.

### SUMMARY OF THE INVENTION

It is a feature of an embodiment of the invention to provide improved compositions and delivery systems to administer acidic pharmaceutical drugs through the skin. It also a feature of an embodiment of the invention to provide methods of making the compositions, as well as methods of administering the compositions to a patient in need thereof.

In accordance with these and other features of various embodiments of the invention, there is provided a topical composition including a molecular complex formed between an acidic pharmaceutical drug and at least one functional substance selected from the group consisting of an alkaline amino acid, an amino acid amide, an amino acid ester, a related alkaline amino acid, or combinations thereof.

In accordance with additional features of embodiments of the invention, there is provided a method of forming a molecular complex between an acidic pharmaceutical drug and at least one functional substance. The method involves dissolving or suspending the acidic pharmaceutical drug (present as an alkali salt) in an appropriate medium, together with an acid to form a free acid of the pharmaceutical drug, and then optionally separating the free acid from the medium. The method further includes adding at least one functional substance to the free acid in a reaction medium to form a molecular complex.

In accordance with an additional feature of an embodiment of the invention, there is provided a method of administering an acidic pharmaceutical drug to a patient in need thereof, comprising topically applying a molecular complex formed between an acidic pharmaceutical drug and at least one functional substance selected from an amino acid, an amino acid amide, an amino acid ester, a related amino acid, or combinations thereof. The molecular complex includes a therapeutically effective amount of the acidic pharmaceutical drug.

### DETAILED DESCRIPTION OF PREFERRED EMBODIMENTS

Embodiments of the invention are not limited to the particular methodology, protocols, and reagents described in the preferred embodiments, as these may vary. It also is to be understood that the terminology used herein is for the purpose of describing particular embodiments only, and is not intended to limit the scope of any embodiment of the present invention.

Throughout this disclosure, the singular forms “a,” “an,” and “the” include plural reference unless the context clearly dictates otherwise. Thus, for example, a reference to “an acidic pharmaceutical drug” includes a plurality of such drugs, and a reference to “a functional substance” is a reference to one or more functional substances and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the various molecules, drugs, delivery systems, and methodologies that are reported in the publications and that might be used in connection with the invention. Nothing herein is to be construed as an admission that the invention is not entitled to antedate such disclosures by virtue of prior invention.

The expression “pharmaceutically effective amount” is used herein to denote a quantity of pharmaceutical that is known to be effective to achieve the desired and known result of the drug. The actual amount contained in the molecular complex, likely will vary from the pharmaceutically effective amount, since some of the drug may not completely penetrate the skin together with the complex. Using the guidelines provided herein, those skilled in the art are capable of determining the pharmaceutically acceptable amount of acidic pharmaceutical drugs described herein, and to use the requisite amount in the molecular complex so that the pharmaceutically acceptable amount is delivered to the subject in need thereof.

Embodiments of the invention relate to molecular complexes, methods of making the molecular complexes, and methods of administering the molecular complexes. The molecular complexes preferably are formed between an acidic, more preferably a weakly acidic pharmaceutical drug, and a functional substance. The acidic pharmaceutical drugs preferably are organic chemical substances which have a pH of below 7.0 after it is dissolved in an aqueous solution, and which contains a carboxyl, sulfuric, nitric, N-oxide and/or carbonylimino group in the molecule. The functional substance preferably includes a basic amino acid, amino acid amide, amino acid ester and related alkaline amino acid with molecular weight of between 50 and 500.

In human skin, the stratum corneum consists of keratin-enriched corneocytes that are embedded in a lipid matrix and are resistant to penetration by ionic compounds or large molecules having a molecular weight of 800 or larger. Some acidic pharmaceutical drugs are commercially available for oral adiminstration in the form of a salt with an organic or inorganic alkali such as sodium hydroxide or potassium hydroxide because the free acid is chemically unstable. When such inorganic salts are incorporated into a topical formulation, the drug exists as a negatively charged anion and cannot or minimally penetrates the stratum corneum of the skin. The drug as fully ionized anion is not in bioavailable form. The reason is that the inorganic alkali used for stabilization and isolation of the drug is a strong alkali and the drug molecule is fully ionized by such alkali.

The present inventors have discovered a relatively simple process for converting the inorganic salt of an acidic pharmaceutical drug into a molecular complex that provides the requisite bioavailability and therapeutic efficacy. In accordance with a preferred embodiment of the method, an inorganic salt of an acidic pharmaceutical drug is reacted with an equimolar amount of acid such as hydrochloric acid to generate the free acid of the drug. Preferably, the reaction vessel is cooled externally with an ice-water bath. If the free acid of the drug is separated from the reaction medium as a water insoluble solid or liquid, the free acid of the drug may be isolated by filtration or decantation using techniques known in the art. Optionally, a function substance may be directly added to form a molecular complex with the free acid.

The free acid (either separated or not from the initial reaction medium) of the drug then is reacted with a functional substance to form a molecular complex. The formation of the molecular complex preferably is indicated when a desired pH is achieved (e.g., from about 2 to about 7). At this point, most of the drug molecules form a molecular complex with the functional substance under dipolar/dipolar, dipolar/ionic, and ionic/ionic attractive forces. If the free acid were separated from the initial reaction medium, the free acid then preferably would be suspended in water and reacted with the functional substance to form the molecular complex. The molecular complex so formed is more bioavailable and therapeutically more effective and less irritating for topical treatment of various cutaneous indications including skin and nail diseases.

The expression “molecular complex” as used throughout this description to define the formation of a molecular complex between an acidic pharmaceutical drug and the functional substance denotes a complex based on three attracting forces. These three attracting forces in increasing strength are: (a) dipolar/dipolar; (b) dipolar/ionic; and (c) ionic/ionic.

When a composition containing the above molecular complex is topically applied to the skin, the drug molecules having a dipolar/dipolar attracting force will penetrate the skin first, followed by the drug molecules having dipolar/ionic attracting forces. The drug molecules having the ionic/ionic attracting forces are typically in salt form and therefore are not generally bioavailable for penetration into the skin. However, when more drug molecules with a dipolar/dipolar attracting force penetrate into the skin, the drug molecules having ionic/ionic attractive forces will become dipolar/ionic and can penetrate the skin when the drug anion is converted to a free acid due to the dissociation equilibrium shift (Henderson-Hasselbalch Equation). Thus, most drug molecules become bioavailable in the molecular complex with at least one functional substance. Typically, the molecular complex formed between a weakly acidic drug and a functional substance by the above intermolecular attractive forces is present in a topical formulation that contains water, alcohol, propylene glycol, butylene glycol or the like.

The molar ratio of acidic drug to functional substance preferably ranges from about 1:0.1 to about 1:40, with a preferred range of from about 1:0.5 to about 1:5. The formation of a molecular complex is more than or beyond the neutralization reaction between an alkali and an acid because the extra functional group(s), e.g., hydroxyl group(s), participate in the formation of molecular complex through intermolecular attracting forces. The inventors believe that all acidic pharmaceutical drugs that have carboxyl, sulfuric, nitric, N-oxide and/or carbonylimino groups in the molecule can form a molecular complex with a functional substance to provide a molecular complex with improved bioavailabilty and improved delivery into the skin and nail plate.

Topical formulations containing molecular complexes are therapeutically effective with less or no stinging or irritations to cutaneous tissues. For example, azelaic acid is used at 15 to 20% concentration in cream formulation for acne, rosacea and melasma. One major side effect of topical treatment with azelaic acid is skin irritation. The optimized azelaic acid composition comprising a molecular complex with L-arginine has been found to be therapeutically effective without skin irritation.

The expression “acidic pharmaceutical drug” or “weakly acidic pharmaceutical drug” denotes a pharmaceutical agent that is an acid in its native form, but typically is administered in its salt form, and that has a pharmaceutical effect. Representative weakly acidic pharmaceutical drugs are listed as follows: acetaminophen, acetaminosalol, acetazolamide, acitretin, acrivastine, ampicillin, arbutin, azelaic acid, benzoyl peroxide, caffeic acid, chlorothiazide, chlorpropamide, ciclopirox, ciprofloxacin, cromolyn, ethacrynic acid, ferulic acid, furosemide, hydroquinone, ibuprofen, kojic acid, methotrexate, penicillamine, penicillins, pentobarbital, phenobarbital, phenytoin, perindopril, propylthiouracil, rabeprazole, retinoic acid, risedronic acid, salicylic acid, sulfacetamide, sulfabenz, sulfabenzamide, sulfabromomethazine. sulfachlorpyridazine, sulfacytine, sulfadimethoxine, sulfadoxine, sulfaguanole, sulfalene, sulfamethizole, sulfamethoxazole, sulfapyrazine, sulfapyridine, sulfasalazine, sulfasomizole, sulfathiazole, theophylline, thioctic acid (lipoic acid), 6,8-dimercaptooctanoic acid (dihydrolipoic acid), tolbutamide, triclosan, urocanic acid, ursodiol, and warfarin.

The functional substances which are useful for the formation of molecular complexes may be divided into four basic groups: namely, (A) alkaline amino acids, (B) amino acid amides, (C) amino acid esters and (D) related amino Acids.

A. Alkaline Amino Acids

An alkaline amino acid is an amino acid that has an extra basic group such as amino, imino and/or guanido groups so that the molecule is alkaline in nature. The functional substances that are useful for the present invention include arginine, histidine, lysine, omithine and tryptophan. The secondary functional group(s) of these alkaline amino acids include the carboxyl and the extra amino, imino and/or guanido groups which can form intermolecular attracting forces with acidic pharmaceutical drugs.

B. Amino Acid Amides

Amino acid amides that are useful as functional substances may be represented by the following generic structure:

RCH(NH₂)CONH₂

where R is H, an alkyl, aralkyl or aryl group having 1 to 14 carbon atoms, and in addition R may carry OH, SH, SCH₃, NH₂, CONH₂, NHCONH₂, NH(C═NH)NH₂, imidazole, pyrrole or other heterocyclic group. The H attached to a carbon atom may be substituted by 1, F, Cl, Br, OH or alkoxy group having 1 to 9 carbons. The amino acid amides may be isomeric such as D, L, or DL-alaninamide or non-isomeric glycinamide. Among commonly known amino acid amides prolinamide cannot be represented by the above generic structure because the alpha amino group is part of the heterocyclic pyrrole ring.

Representative amino acid amides include alaninamide, β-alaninamide, γ-aminobutanoamide, β-aminoisobutanoamide, argininamide, aspartic diamide, asparaginamide, citrullinamide, cysteinamide, glycinamide, glutamic diamide, glutaminamide, histidinamide, homocysteinamide, homoserinamide, isoleucinamide, leucinamide, lysinamide, methioninamide, omithinamide, phenylalaninamide, phenylglycinamide, 4-hydroxyphenylglycinamide, prolinamide, serinamide, threoninamide, tryptophanamide, tyrosinamide, valinamide, and mixtures thereof. The secondary functional group(s) of these amino acid amides include OH, SH, SCH₃, CONH₂, NHCONH₂, NH(C═NH)NH₂, imidazole and extra NH₂ or CONH₂ group that can form intermolecular attracting forces with acidic pharmaceutical drugs.

C. Amino Acid Esters

Amino acid esters which are useful as functional substances may be represented by the following generic structure:

RCH(NH₂)COOR₁

where R is H, an alkyl, aralkyl or aryl group having 1 to 14 carbon atoms; R₁ is an alkyl, aralkyl or aryl group having 1 to 9 carbon atoms; and in addition R may carry OH, SH, SCH₃, NH₂, CONH₂, NHCONH₂, NH(C=NH)NH₂, imidazole, pyrrole or other heterocyclic group. The H attached to a carbon atom may be substituted by 1, F, Cl, Br, OH or alkoxy group having 1 to 9 carbons. The amino acid esters may be isomeric such as D, L, or DL-tyrosine ethyl ester or non-isomeric glycine ethyl ester. Among commonly known amino acid esters, proline esters cannot be represented by the above generic structure because the alpha amino group is part of the heterocyclic pyrrole ring.

The following are representative amino acid esters.

(1) methyl alaninate, ethyl alaninate, propyl alaninate and isopropyl alaninate

(2) methyl β-alaninate, ethyl β-alaninate, propyl β-alaninate and isopropyl β-alaninate

(3) methyl γ-aminobutanoate, ethyl γ-aminobutanoate, propyl γ-aminobutanoate and isopropyl γ-aminobutanoate

(4) methyl β-aminoisobutanoate, ethyl β-aminoisobutanoate, propyl β-aminoisobutanoate and isopropyl β-aminoisobutanoate

(5) methyl argininate, ethyl argininate, propyl argininate and isopropyl argininate

(6) dimethyl aspartate, diethyl aspartate, dipropyl aspartate and diisopropyl aspartate

(7) methyl asparaginate, ethyl asparaginate, propyl asparaginate and isopropyl asparaginate

(8) methyl citrullinate, ethyl citrullinate, propyl citrullinate and isopropyl citrullinate

(9) methyl cysteinate, ethyl cysteinate, propyl cysteinate and isopropyl cysteinate

(10) methyl glycinate, ethyl glycinate, propyl glycinate and isopropyl glycinate

(11) dimethyl glutamate, diethyl glutamate, dipropyl glutamate and diisopropyl glutamate

(12) methyl glutaminate, ethyl glutaminate, propyl glutaminate and isopropyl glutaminate

(13) methyl histidinate, ethyl histidinate, propyl histidinate and isopropyl histidinate

(14) methyl homocysteinate, ethyl homocysteinate, propyl homocysteinate and isopropyl homocysteinate

(15) methyl homoserinate, ethyl homoserinate, propyl homoserinate and isopropyl homoserinate

(16) methyl isoleucinate, ethyl isoleucinate, propyl isoleucinate and isopropyl isoleucinate

(17) methyl leucinate, ethyl leucinate, propyl leucinate and isopropyl leucinate

(18) methyl lysinate, ethyl lysinate, propyl lysinate and isopropyl lysinate

(19) methyl methioninate, ethyl methioninate, propyl methioninate and isopropyl methioninate

(20) methyl omithinate, ethyl omithinate, propyl omithinate and isopropyl omithinate

(21) methyl phenylalaninate, ethyl phenylalaninate, propyl phenylalaninate and isopropyl phenylalaninate

(22) methyl phenylglycinate, ethyl phenylglycinate, propyl phenylglycinate and isopropyl phenylglycinate

(23) methyl 4-hydroxyphenylglycinate, ethyl 4-hydroxyphenylglycinate, propyl 4-hydroxyphenylglycinate and isopropyl 4-hydroxyphenylglycinate

(24) methyl prolinate, ethyl prolinate, propyl prolinate and isopropyl prolinate

(25) methyl serinate, ethyl serinate, propyl serinate and isopropyl serinate

(26) methyl threoninate, ethyl threoninate, propyl threoninate and isopropyl threoninate

(27) methyl tryptophanate, ethyl tryptophanate, propyl tryptophanate and isopropyl tryptophanate

(28) methyl tyrosinate, ethyl tyrosinate, propyl tyrosinate and isopropyl tyrosinate; and

(29) methyl valinate, ethyl valinate, propyl valinate and isopropyl valinate.

The secondary functional group(s) of these amino acid esters include OH, SH, SCH₃, CONH₂, NHCONH₂, NH(C═NH)NH₂, imidazole and extra NH₂ and/or COOR group which can form intermolecular attracting forces with acidic pharmaceutical drugs.

D. Related Alkaline Amino Acids

Related amino acids include diamino acids of non-protein components, other related or derived amino acids, their amides and esters which can form molecular complexes with acidic pharmaceutical drugs. The related amino acids include the following: creatinine, 2,3-diaminopropanoic acid; 2,3-diaminopropanoamide; 2,3-diaminopropanoic acid esters; 2,3-diaminobutanoic acid; 2,3-diaminobutanoamide; 2,3-diaminobutanoic acid esters; 2,4-diaminobutanoic acid; 2,4-diaminobutanoamide; 2,4-diaminobutanoic acid esters; 3,4-diaminobutanoic acid; 3,4-diaminobutanoamide; 3,4-diaminobutanoic acid esters; 2,3-diaminopentanoic acid; 2.3-diaminopentanoamide; 2,3-diaminopentanoic acid esters; 2,4-diaminopentanoic acid; 2,4-diaminopentanoamide; 2,4-diaminopentanoic acid esters; 2,5-diaminopentanoic acid; 2,5-diaminopentanoamide; 2,5-diaminopentanoic acid esters; N^{ω}-methylarginine; N^{ω}-dimethylarginine; N^{ω},N^{ω′}-dimethylarginine; N^{T}-methylhistidine; N^{ε}-methyllysine; N^{ε}-dimethyllysine; N^{ε}-trimethyllysine; N^{ε}-trimethyl-δ-hydroxylysine;δ-hydroxylysine; 2-amino-3-methylaminopropanoic acid; canaline; canavanine; 2,4-diamino-3-methylbutanoic acid; 2,3-diaminobutanoic acid; 2,4-diaminobutanoic acid; 2,4-diaminovaleric acid; 4,5 dihydroxyornithine; N^{G},N^{G}-dimethylarginine; N^{G}, N^{G}-dimethylarginine; N⁶-dimethyllysine; homoarginine; 4-hydroxylysine; 5-hydroxylysine; 4-hydroxyarginine; 4-hydroxyhomoarginine; 4-hydroxyornithine; hypusine; indospicine; 2-methylarginine; N⁵-methylornithine; N^{G}-methylarginine; N⁶-methyllysine and oxalysine.

The secondary functional group(s) of these amino acids, their amides and esters, and related compounds include OH, SH, SCH₃, CO, CONH₂, NHCONH₂, NH(C═NH)NH₂, imidazole, pyrrole and extra NH₂, COOR and/or CONH₂ group which can form intermolecular attracting forces with acidic pharmaceutical drugs.

The molecular complex formed from an acidic pharmaceutical drug and a functional substance has been found to provide optimal bioavailability for topical treatment of various cutaneous disorders including cosmetic conditions and dermatological diseases. The functional substances include basic amino acids, amino acid amides, amino acid esters and related amino acids with molecular weight of between 50 and 500 and include for example, arginine, lysine, histidine, tryptophan, omithine, glycinamide and glycine ethyl ester.

The molecular complex composition also may contain other pharmaceutical or topical agents to further expand the utilities for maximal therapeutic efficacies, such as in combination with N-acetyl aldosamines or N-acetylamino acids as disclosed in U.S. Pat. No. 6,159,485, or with oligosaccharide aldonic acids in U.S. Pat. No. 6,335,023. The disclosures of each of these patents are incorporated by reference herein in their entirety.

The pharmaceutical and other topical agents that may be incorporated into molecular complex compositions include those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants and hormones.

Representative pharmaceutical and topical agents include aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, bacitracin, bemegride, beclomethasone dipropionate, benzophenone, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, clemastine, clindamycin, clioquinol, clobetasol propionate, clotrimazole, coal tar, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroxyzine, ichthammol, imiquimod, indomethacin, ketoconazole, ketoprofen, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocalne, procaine, promethazine propionate, propranolol, pseudoephedrine, pyrethrin, pyrilamine, resorcinol, retinal, retinol, retinyl acetate, retinyl palmitate, salicylamide, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, N-acetyl-prolinamide, N-acetyl-lysine, N-acetyl-omithine, N-acetyl-glucosamine, and mixtures thereof.

Another example of cosmetic or other agents that may be combined with the molecular complex include hydroxyacids, ketoacids and related compounds. Examples of hydroxy acids include hydroxymonocarboxylic acids, hydroxydicarboxylic acids, 2-hydroxycarboxylic acids, other hydroxycarboxylic acids, 2-ketocarboxylic acids and related compounds. See, for example, U.S. Pat. Nos. 5,422,370, 5,547,988, 5,470,880, and 5,385,938, the disclosures of which are incorporated by reference herein in their entirety. The hydroxy acids may exist as a free acid, an ester, a lactone, in salt form with an organic base or an inorganic alkali, and as stereoisomers. Representative examples of hydroxy acids and related compounds include glycolic acid, mandelic acid, lactic acid, tropic acid, methyllactic acid, tartaric acid, citric acid, glucuronic acid, ribonic acid, gluconolactone, ribonolactone, gycolyl glycollate, lactyl lactate, trilactic acid and polylactic acid.

A particularly preferred process for preparing the molecular complex of the invention includes dissolving an acidic pharmaceutical drug 0.1 mole in salt form in water (50 ml), and an acid, preferably about 4N HCI 25 ml, is added slowly with stirring while the reaction flask is cooled externally in an ice-water bath. The free acid drug is formed instantly and is usually separated as precipitate or oily product. The precipitate can be isolated by filtration and washed with water and dried. The oily product can be isolated and washed with water using a separatory funnel.

To prepare a typical molecular complex composition, the above free acid drug (0.1 mole) isolated as precipitate or oily liquid preferably is suspended in water 50 ml, and a functional substance, preferably arginine or lysine, then is added with stirring. Alternatively, other solvents such as ethanol, propylene glycol, butylene glycol, etc. may be added to water solution before or after the formation of molecular complex. The formation of a molecular complex can be evidenced by raising pH, and the reaction is completed when there is no more change in the pH. The concentration of the functional substance preferably may vary from 0.1 to about 10 moles with preferred ranges of from about 0.2 to about 3 moles when one mole of an acidic pharmaceutical drug is used. The final pH of the composition containing the molecular complex may range from about 2.0 to about 9.0, more preferably from about 3.0 to about 8.0.

To prepare a synergistic or synergetic composition, a pharmaceutical or other topical agent preferably is added directly or first dissolved in water or other solvent, and then added into a composition containing the molecular complex.

Other forms of compositions such as solution, lotion, cream, ointment, gel etc. for topical delivery of the molecular complex between an acidic drug and a functional substance of the instant invention are readily prepared or formulated by those skilled in the art.

Because the molecular complex should be effective in permitting the release of the drug through the skin, it is preferred that the molecular weight of the functional substance be within the range of from about 50 to about 800. It is more preferred that the molecular weight be within the range of from about 60 to about 700, and most preferred within the range of from about 70 to about 500.

The present inventors also have discovered that compositions comprising a molecular complex of preferred embodiments of the present invention are topically effective for the general care of skin, hair and nail; nasal, oral and vaginal mucosa. The compositions are useful in a variety of methods, including: treatment, healing and prevention of cosmetic conditions and dermatological indications, as well as cosmetic and clinical signs of changes associated with intrinsic or extrinsic aging; the damages caused by extrinsic factors such as sunlight, air pollution, wind, cold, dampness, heat, chemicals, smoke, cigarette smoking, and radiations including electromagnetic radiations and ionizing radiations. The compositions also are useful for reducing and soothing mucosa and skin erythema, inflammation or reaction caused by internal or external factors.

General cosmetic conditions and dermatological indications that can be treated using the molecular complexes of various embodiments of the invention include: disturbed keratinization, inflammation, defective syntheses of dermal components, and changes associated with intrinsic and extrinsic aging of skin, nail and hair. Particular conditions and indications include: dryness or looseness of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; dermatoses; eczema; psoriasis; pruritus; warts; herpes; age spots; lentigines; melasmas; blemished skin; hyperkeratoses; hyperpigmented or hypopigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubticants and luster; dull and older-looking skin, nail and hair; fragility and splitting of nail and hair, or used as to lighten the skin.

Specific skin changes associated with aging include, but are not limited to, progressive thinning of skin, fragile skin, deepening of skin lines and fine lines, wrinkles including fine and coarse wrinkles, lusterless skin surface, coarse and uneven skin, loss of skin elasticity and recoilability, blemished and leathery skin, loss of skin lubricating substances, increased numbers of blotches and mottles, nodules, pre-cancerous lesions, pigmented spots and mottled skin, changes in qualities and quantities of collagen and elastic fibers, solar elastosis, decrease in collagen fibers, diminution in the number and diameter of elastic fibers in the papillary dermis, atrophy of the dermis, stretch marks, reduction in subcutaneous adipose tissue and deposition of abnormal elastic materials in the upper dermis, yellowing skin, telangiectatic skin and older-looking skin.

The concentration of the acidic pharmaceutical drug may range anywhere from 0.01 to 99.9%, with preferred concentration of from about 0.1 to 50% and with more preferred concentration of from about 1 to 25% by weight of the total composition. Other advantageous concentration ranges provide a concentration of at least 3%, 4% or 5% of the acidic pharmaceutical drug. Higher concentrations of an acidic pharmaceutical drug in the ranges of 40%, 50%, 60% or more also can be employed, depending on the desired end use. Thus, acceptable ranges of an acidic pharmaceutical drug will be from about 1%, 2%, 3%, 4% or 5% at the minimum, to about 95% at maximum, and within that range will be ranges of from about 1% to about 5%, from about 5% to about 10%, from about 10% to about 20%, from about 20% to about 40%, from about 40% to about 60%, from about 60% to about 80%, from about 80% to about 95%. These weights are based on the weight of the total composition.

The concentration of the functional substance or combinations thereof, may range from 0.01 to 99.9%. Advantageous concentrations will comprise at least 0.2% functional substance, and typically at least about 1% or 2% of functional substance. Other advantageous concentration ranges provide at least being at least 3%, 4% or 5% of functional substance. Higher concentrations of a functional substance in the ranges of 40%, 50%, 60% or more also can be employed. Thus, typical ranges of a functional substance will be from about 1%, 2%, 3%, 4% or 5% at the minimum to 99.9% at maximum, and within that range will be ranges of from about 5% to about 10%, from about 10% to about 20%, from about 20% to about 40%, from about 40% to about 60%, from about 60% to about 80%, from about 80% to about 99.9%. These weights are based on the weight of the total composition.

To prepare a topical composition in lotion, cream or ointment form, the above aqueous mixture containing the molecular complex preferably is mixed in a conventional manner with a commonly available lotion, cream or ointment base. A topical composition of the instant invention may also be formulated in a gel form. A typical gel composition can be prepared by the addition of a gelling agent such as methyl cellulose, ethyl cellulose, hydroxyethylcellulose, hydroxypropylcellulose, hydroxypropylmethylcellulose, carbomer or ammonium glycyrrhizate to a solution mixture containing the molecular complex. The preferred concentration of the gelling agent may range from 0.1 to 4 percent by weight of the total composition.

The following are illustrative examples of formulations and the test results, and are not limiting. Therefore, any of the aforementioned acidic drugs and functional substances can be substituted according to the teachings of this invention in the following examples.

### EXAMPLE 1

A typical molecular complex formed between an acidic drug and a functional substance was prepared as follows. Azelaic acid 9.4 g (50 mmole) was dissolved in 86.2 ml solution prepared from water 40 parts, ethanol 40 parts and propylene glycol 20 parts by volume. L-Arginine 4.4 g (25 mmole) was added to form a molecular complex, and the reaction mixture maintained until the solution changed pH from 3.2 to 5.1. The solution thus prepared contained 9.4% azelaic acid in molecular complex with 4.4% L-arginine. The azelaic acid composition comprising the molecular complex with L-arginine has been found to be less or non-irritating to the skin and should be therapeutically beneficial for topical treatment of acne vulgaris and rosacea.

### EXAMPLE 2

Azelaic acid 9.4 g (50 mmole) was dissolved in 87 ml solution prepared from water 40 parts, ethanol 40 parts and propylene glycol 20 parts by volume. L-Lysine 3.65 g (25 mmole) was added to form a molecular complex and the reaction mixture maintained until the solution changed from pH of 3.2 to 5.4. The solution thus prepared contained 9.4% azelaic acid in molecular complex with 3.7% L-lysine. A male subject, age 35, having oily skin on the face topically applied the above azelaic solution on his nose. After topical application, there were no signs of skin irritations. A female subject, age 34, having sensitive skin topically applied the above azelaic acid solution on her face. There were no signs of skin irritations from the above azelaic acid formulation. The azelaic acid composition comprising the molecular complex with L-lysine should be therapeutically beneficial for topical treatment of acne vulgaris and rosacea.

### EXAMPLE 3

Ciclopirox 2.1 g (10 mmole) was dissolved in 98 ml solution prepared from water 40 parts, ethanol 40 parts and propylene glycol 20 parts by volume. L-Arginine 0.35 g (2 mmole) was added to form a molecular complex and the reaction mixture maintained until the pH of the solution changed to pH 7.1. The solution thus prepared contained 2.1% ciclopirox in molecular complex with 0.35% L-arginine. The ciclopirox composition comprising the molecular complex with L-arginine should be therapeutically beneficial without irritation for topical treatment of fungal infections of the skin or nails.

### EXAMPLE 4

Ciclopirox 2.1 g (10 mmole) was dissolved in 97 ml solution prepared from water 40 parts, ethanol 40 parts and propylene glycol 20 parts by volume. L-Arginine 0.87 g (5 mmole) was added to form a molecular complex and the reaction mixture maintained until the pH of the solution changed to pH 7.8. The solution thus prepared contained 2.1% ciclopirox in molecular complex with 0.87% L-arginine. The ciclopirox composition comprising the molecular complex with L-arginine should be therapeutically beneficial without irritation for topical treatment of fungal infections of skin or nails.

### EXAMPLE 5

Ciclopirox 3.1 g (15 mmole) was dissolved in 96 ml solution prepared from water 40 parts, ethanol 40 parts and propylene glycol 20 parts by volume. Creatinine 0.56 g (5 mmole) was added to form a molecular complex and the mixture maintained until the pH of the solution changed to pH 6.6. The solution thus prepared contained 3.1% ciclopirox in molecular complex with 0.56% creatinine. The ciclopirox composition comprising the molecular complex with creatinine should be therapeutically beneficial without irritation for topical treatment of fungal infections of the skin or nails.

### EXAMPLE 6

Ciclopirox 4.1 g (20 mmole) was dissolved in 95 ml solution prepared from water 40 parts, ethanol 40 parts and propylene glycol 20 parts by volume. L-Arginine 0.87 g (5 mmole) was added to form a molecular complex and the mixture maintained until the pH of the solution changed to pH 7.3. The solution thus prepared contained 4.1% ciclopirox in molecular complex with 0.87% L-arginine. The ciclopirox composition comprising the molecular complex with L-arginine should be therapeutically beneficial without irritation for topical treatment of fungal infections of the skin or nails.

### EXAMPLE 7

Ciclopirox 8.28 g (40 mmole) was dissolved in 89 ml solution prepared from water 40 parts, ethanol 40 parts and propylene glycol 20 parts by volume. L-Lysine 2.92 g (20 mmole) was added to form a molecular complex and the mixture maintained until the pH of the solution changed to pH 7.9. The solution thus prepared contained 8.3% ciclopirox in molecular complex with 2.9% L-lysine.

A male subject, age 72, having fungal infections on the left toe nails for several months topically applied the above formulation once daily on the infected nail plates. There were no signs of irritations after topical applications of the above molecular complex formulation. After two months of topical treatment, there was no clinical signs of fungal infections and the toe nails continued to grow normally. This result shows that the molecular complex formed between ciclopirox and L-lysine is non-irritating and therapeutically effective for topical treatment of fungal infections.

### EXAMPLE 8

Salicylic acid 6.9 g (50 mmole) was dissolved in 92 ml solution prepared from water 40 parts, ethanol 40 parts and propylene glycol 20 parts by volume. L-Lysine 1.46 g (10 mmole) was added to form a molecular complex and the mixture maintained until the pH of the solution changed to pH 3.2. The solution thus prepared contained 6.9% salicylic acid in molecular complex with 1.5% L-lysine. A male subject, age 70, having fissured calluses on his feet topically applied twice daily the above salicylic acid solution for a few days. After a few days of topical treatment, the calluses became soft and thinner without any signs of skin irritations. This result indicated that salicylic acid composition comprising the molecular complex with L-lysine was therapeutically effective for topical treatment of hyperkeratotic conditions.

### EXAMPLE 9

Salicylic acid 6.9 g (50 mmole) was dissolved in 92 ml solution prepared from water 40 parts, ethanol 40 parts and propylene glycol 20 parts by volume. L-Arginine 1.74 g (10 mmole) was added to form a molecular complex and the mixture maintained until the pH of the solution changed to pH 3.2. The solution thus prepared contained 6.9% salicylic acid in molecular complex with 1.7% L-arginine. A female subject, age 34, having adolescent acne on her face, topically applied twice daily the above solution for several weeks. There were no signs of skin irritations from topical applications of the solution. After several weeks of topical treatment, most lesions became less inflamed and gradually improved. This result showed that salicylic acid composition comprising the molecular complex with L-arginine was therapeutically effective without irritation for topical treatment of acne.

### EXAMPLE 10

Salicylic acid 1.38 g (10 mmole) was dissolved in 97 ml solution prepared from water 40 parts, ethanol 40 parts and propylene glycol 20 parts by volume. Creatinine 1.13 g (10 mmole) was added to form a molecular complex and the mixture maintained until the pH of the solution changed to pH 4.1. The solution thus prepared contained 1.4% salicylic acid in molecular complex with 1.1% creatinine. The salicylic acid composition comprising the molecular complex with creatinine should be therapeutically beneficial for topical treatment of acne.

### EXAMPLE 11

A typical process of converting an acidic pharmaceutical drug from a metallic salt to a free acid form is carried out as follows. Methotrexate disodium salt 3.75 g (7.5 mmole) was dissolved in water 150 ml, and 1 N HCI 7.5 ml was added with stirring and cooling externally with ice-water bath. The conversion process was completed as shown by the precipitation of yellowish product and the change of pH from 6.9 to 4.8. The yellowish methotrexate free acid could be isolated by filtration or centrifugation. Alternatively, the mixture could be directly used for the preparation of a molecular complex as follows.

Methotrexate free acid 1.73 g (3.8 mmole) was suspended in water 90 ml, and L-arginine 0.6 g (3.4 mmole) was added with stirring. The formation of a molecular complex between methotrxate and L-arginine was completed as shown by the dissolution of methotrexate and the change of pH from 4.8 to 6.0. After addition of propylene glycol 20 ml, the final formulation contained a molecular complex between 3.8 mmole methotrxate and 3.4 mmole L-arginine with pH 6.1.

### EXAMPLE 12

Methotrexate free acid 1.73 g (3.8 mmole) was suspended in water 90 ml, propylene glycol 30 ml and ethanol 20 ml, and L-lysine 0.4 g (2.7 mmole) was added with stirring to form a molecular complex. The formation of a molecular complex between methotrxate and L-lysine was completed as shown by the dissolution of methotrexate and the change of pH from 4.8 to 6.2. The formulation thus prepared contained a molecular complex between 3.8 mmole methotrxate and 2.7 mmole L-lysine.

A male subject, age 85, having chronic plaque psoriasis had a 1 cm² area of involved skin in his right forearm treated with the above molecular complex formulation under Hays Occlusive Chamber for 24 hours. After 24 hours, the Chamber was removed, and there were no signs of any skin irritations from topical application of the formulation. After one week of no further treatment, the treated site was clinically judged to have approximately 25% improvement. This result shows that methotrexate molecular complex was therapeutically effective for topical treatment of psoriasis.

The invention has been described with reference to particularly preferred examples and embodiments. Those skilled in the art will appreciate that the invention is not limited to these embodiments, but may vary widely using the teachings provided herein, and that the variations and modifications are within the scope of the invention.

What is claimed is:

1. A composition comprising a molecular complex formed between:
an acidic pharmaceutical drug; and
at least one functional substance selected from the group consisting of an alkaline amino acid, an amino acid amide, an amino acid ester, a related alkaline amino acid, or combinations thereof.

2. The composition as claimed in claim 1, wherein the functional substance is an alkaline amino acid.

3. The composition as claimed in claim 2, wherein the alkaline amino acid is selected from the group consisting of arginine, histidine, lysine, ornithine, tryptophan, and mixtures thereof, each of which has an extra basic group selected from amino, imino and/or guanido groups.

4. The composition as claimed in claim 1, wherein the functional substance is an amino acid amide having the following formula:
RCH(NH₂)CONH₂
where R is H, an alkyl, aralkyl or aryl group having 1 to 14 carbon atoms, and in addition R may carry OH, SH, SCH₃, NH₂, CONH₂, NHCONH₂, NH(C═NH)NH₂, imidazole, pyrrole or other heterocyclic group, the H attached to a carbon atom may be substituted by 1, F, Cl, Br, OH or alkoxy group having 1 to 9 carbons, and wherein the amino acid amides may be isomeric such as D, L, or DL or non-isomeric.

5. The composition as claimed in claim 4, wherein the amino acid amide is selected from the group consisting of alaninamide, β-alaninamide, γ-aminobutanoamide, β-aminoisobutanoamide, argininamide, aspartic diamide, asparaginamide, citrullinamide, cysteinamide, glycinamide, glutamic diamide, glutaminamide, histidinamide, homocysteinamide, homoserinamide, isoleucinamide, leucinamide, lysinamide, methioninamide, ornithinamide, phenylalaninamide, phenylglycinamide, 4-hydroxyphenylglycinamide, prolinamide, serinamide, threoninamide, tryptophanamide, tyrosinamide, valinamide, and mixtures thereof.

6. The composition as claimed in claim 5, wherein the amino acid amide contains a secondary functional group selected from the group consisting of OH, SH, SCH₃, CONH₂, NHCONH₂, NH(C═NH)NH₂, imidazole and extra NH₂ or CONH₂ group that can form intermolecular attracting forces with acidic pharmaceutical drugs.

7. The composition as claimed in claim 1, wherein the functional substance is an amino acid ester having the following general formula:
RCH(NH₂)COOR₁
where R is H, an alkyl, aralkyl or aryl group having 1 to 14 carbon atoms; R₁ is an alkyl, aralkyl or aryl group having 1 to 9 carbon atoms; and in addition R may carry OH, SH, SCH₃, NH₂, CONH₂, NHCONH₂, NH(C═NH)NH₂, imidazole, pyrrole or other heterocyclic group, the H attached to a carbon atom may be substituted by 1, F, Cl, Br, OH or alkoxy group having 1 to 9 carbons, and where the amino acid esters may be isomeric such as D, L, or DL or non-isomeric.

8. The composition as claimed in claim 7, wherein the amino acid ester is selected from the group consisting of:
(1) methyl alaninate, ethyl alaninate, propyl alaninate and isopropyl alaninate;
(2) methyl β-alaninate, ethyl β-alaninate, propyl β-alaninate and isopropyl ˜β-alaninate;
(3) methyl γ-aminobutanoate, ethyl γ-aminobutanoate, propyl γ-aminobutanoate and isopropyl y-aminobutanoate;
(4) methyl β-aminoisobutanoate, ethyl β-aminoisobutanoate, propyl β-aminoisobutanoate and isopropyl β-aminoisobutanoate;
(5) methyl argininate, ethyl argininate, propyl argininate and isopropyl argininate;
(6) dimethyl aspartate, diethyl aspartate, dipropyl aspartate and diisopropyl aspartate;
(7) methyl asparaginate, ethyl asparaginate, propyl asparaginate and isopropyl asparaginate;
(8) methyl citrullinate, ethyl citrullinate, propyl citrullinate and isopropyl citrullinate;
(9) methyl cysteinate, ethyl cysteinate, propyl cysteinate and isopropyl cysteinate;
(10) methyl glycinate, ethyl glycinate, propyl glycinate and isopropyl glycinate
(11) dimethyl glutamate, diethyl glutamate, dipropyl glutamate and diisopropyl glutamate;
(12) methyl glutaminate, ethyl glutaminate, propyl glutaminate and isopropyl glutaminate;
(13) methyl histidinate, ethyl histidinate, propyl histidinate and isopropyl histidinate;
(14) methyl homocysteinate, ethyl homocysteinate, propyl homocysteinate and isopropyl homocysteinate;
(15) methyl homoserinate, ethyl homoserinate, propyl homoserinate and isopropyl homoserinate;
(16) methyl isoleucinate, ethyl isoleucinate, propyl isoleucinate and isopropyl isoleucinate;
(17) methyl leucinate, ethyl leucinate, propyl leucinate and isopropyl leucinate;
(18) methyl lysinate, ethyl lysinate, propyl lysinate and isopropyl lysinate;
(19) methyl methioninate, ethyl methioninate, propyl methioninate and isopropyl methioninate;
(20) methyl omithinate, ethyl omithinate, propyl omithinate and isopropyl omithinate;
(21) methyl phenylalaninate, ethyl phenylalaninate, propyl phenylalaninate and isopropyl phenylalaninate;
(22) methyl phenylglycinate, ethyl phenylglycinate, propyl phenylglycinate and isopropyl phenylglycinate;
(23) methyl 4-hydroxyphenylglycinate, ethyl 4-hydroxyphenylglycinate, propyl 4-hydroxyphenylglycinate and isopropyl 4-hydroxyphenylglycinate;
(24) methyl prolinate, ethyl prolinate, propyl prolinate and isopropyl prolinate;
(25) methyl serinate, ethyl serinate, propyl serinate and isopropyl serinate;
(26) methyl threoninate, ethyl threoninate, propyl threoninate and isopropyl threoninate;
(27) methyl tryptophanate, ethyl tryptophanate, propyl tryptophanate and isopropyl tryptophanate;
(28) methyl tyrosinate, ethyl tyrosinate, propyl tyrosinate and isopropyl tyrosinate;
(29) methyl valinate, ethyl valinate, propyl valinate and isopropyl valinate; and mixtures thereof.

9. The composition as claimed in claim 8, wherein the amino acid ester contains a secondary functional group selected from the group consisting of OH, SH, SCH₃, CONH₂, NHCONH₂, NH(C═NH)NH₂, imidazole and extra NH₂ and/or COOR groups that can form intermolecular attracting forces with acidic pharmaceutical drugs.

10. The composition as claimed in claim 1, wherein the functional substance is a related amino acid.

11. The composition as claimed in claim 10, wherein the related amino acid is selected from the group consisting of creatinine, 2,3-diaminopropanoic acid; 2,3-diaminopropanoamide; 2,3-diaminopropanoic acid esters; 2,3-diaminobutanoic acid; 2,3-diaminobutanoamide; 2,3-diaminobutanoic acid esters; 2,4-diaminobutanoic acid; 2,4-diaminobutanoamide; 2,4-diaminobutanoic acid esters; 3,4-diaminobutanoic acid; 3,4-diaminobutanoamide; 3,4-diaminobutanoic acid esters; 2,3-diaminopentanoic acid; 2,3-diaminopentanoamide; 2,3-diaminopentanoic acid esters; 2,4-diaminopentanoic acid; 2,4-diaminopentanoamide; 2,4-diaminopentanoic acid esters; 2,5-diaminopentanoic acid; 2,5-diaminopentanoamide; 2,5-diaminopentanoic acid esters; N^{ω}-methylarginine; N^{G}-dimethylarginine; N^{G},N^{G}-dimethylarginine; N^{T}-methylhistidine; N^{ε}-methyllysine; N^{ε}-dimethyllysine; N^{ε}-trimethyllysine; N^{ε}-trimethyl-δ-hydroxylysine;δ-hydroxylysine; 2-amino-3-methylaminopropanoic acid; canaline; canavanine; 2.4-diamino-3-methylbutanoic acid; 2,3-diaminobutanoic acid; 2,4-diaminobutanoic acid; 2,4-diaminovaleric acid; 4,5 dihydroxyornithine; N^{G},N^{G}-dimethylarginine; N^{G},N^{G}-dimethylarginine; N⁶-dimethyllysine; homoarginine; 4-hydroxylysine; 5-hydroxylysine; 4-hydroxyarginine; 4-hydroxyhomoarginine; 4-hydroxyornithine; hypusine; indospicine; 2-methylarginine; N⁵-methylornithine; N^{G}-methylarginine; N⁶-methyllysine, oxalysine, and mixtures thereof.

12. The composition as claimed in claim 11, wherein the related amino acid includes a secondary functional group selected from the group consisting of OH, SH, SCH₃, CO, CONH₂, NHCONH₂, NH(C═NH)NH₂, imidazole, pyrrole and extra NH₂, COOR and/or CONH₂ group which can form intermolecular attracting forces with acidic pharmaceutical drugs.

13. The composition as claimed in claim 1, wherein the functional substance has a molecular weight within the range of from about 50 to about 500.

14. The composition as claimed in claim 1, wherein the functional substance is selected from the group consisting of arginine, lysine, histidine, tryptophan, ornithine, glycinamide, glycine ethyl ester, and mixtures thereof.

15. The composition as claimed in claim 1, wherein the acidic pharmaceutical drug is selected from the group consisting of acetaminophen, acetaminosalol, acetazolamide, acitretin, acrivastine, ampicillin, arbutin, azelaic acid, benzoyl peroxide, caffeic acid, chlorothiazide, chlorpropamide, ciclopirox, ciprofloxacin, cromolyn, ethacrynic acid, ferulic acid, furosemide, hydroquinone, ibuprofen, kojic acid, methotrexate, penicillamine, penicillins, pentobarbital, phenobarbital, phenytoin, perindopril, propylthiouracil, rabeprazole, retinoic acid, risedronic acid, salicylic acid, sulfacetamide, sulfabenz, sulfabenzamide, sulfabromomethazine sulfachlorpyridazine, sulfacytine, sulfadimethoxine, sulfadoxine, sulfaguanole, sulfalene, sulfamethizole, sulfamethoxazole, sulfapyrazine, sulfapyridine, sulfasalazine, sulfasomizole, sulfathiazole, theophylline, thioctic acid (lipoic acid), 6,8-dimercaptooctanoic acid (dihydrolipoic acid), tolbutamide, triclosan, urocanic acid, ursodiol, warfarin, and mixtures thereof.

16. The composition as claimed in claim 1, wherein the molar ratio of the acidic pharmaceutical drug to the functional substance is within the range of from about 1:0.1 to about 1:40.

17. The composition as claimed in claim 1, wherein the molar ratio of the acidic pharmaceutical drug to the functional substance is within the range of from about 1:0.5 to about 1:5.

18. The composition as claimed in claim 1, further comprising pharmaceutical and other topical agents selected from the group consisting of: those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; dipilating agents, and mixtures and combinations thereof.

19. The composition as claimed in claim 1, further comprising one or more additional agents selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, bacitracin, bemegride, beclomethasone dipropionate, benzophenone, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, clemastine, clindamycin, clioquinol, clobetasol propionate, clotrimazole, coal tar, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroxyzine, ichthammol, imiquimod, indomethacin, ketoconazole, ketoprofen, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate 0, permethrin, pheniramine, phenol, phenylephrine, phenylpropanolamine, piperonyl butoxide, podophyllin, podofilox, povidone iodine, pramoxine, prilocalne, procaine, promethazine propionate, propranolol, pseudoephedrine, pyrethrin, pyrilamine, resorcinol, retinal, retinol, retinyl acetate, retinyl palmitate, salicylamide, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, N-acetyl-prolinamide, N-acetyl-lysine, N-acetyl-omithine, N-acetyl-glucosamine, and mixtures thereof.

20. A method of forming a molecular complex between an acidic pharmaceutical drug and at least one functional substance comprising:
dissolving a salt of an acidic pharmaceutical drug in a suitable reaction medium and adding an acid to form a free acid of the pharmaceutical drug;
optionally separating the free acid of the pharmaceutical drug from the reaction medium; and
adding at least one functional substance selected from the group consisting of an alkaline amino acid, an amino acid amide, an amino acid ester, a related alkaline amino acid, or combinations thereof, to the free acid in a suitable reaction medium to form a molecular complex.

21. The method as claimed in claim 20, wherein the free acid of the pharmaceutical drug is separated from the reaction medium.

22. The method as claimed in claim 20, wherein the reaction medium used to form the free acid of the pharmaceutical drug is water.

23. The method as claimed in claim 20, wherein the acid added to the acidic pharmaceutical drug is an inorganic acid.

24. The method as claimed in claim 20, wherein the free acid of the acidic pharmaceutical drug is formed as a precipitate or oily product that then is separated from the reaction medium.

25. The method as claimed in claim 20, wherein the reaction medium used to form the molecular complex is water, and wherein the free acid of the acidic pharmaceutical drug is suspended in the water.

26. The method as claimed in claim 25, wherein the reaction medium additionally comprises a solvent selected from the group consisting of ethanol, propylene glycol, butylene glycol, and mixtures thereof.

27. The method as claimed in claim 20, wherein the molecular complex is formed when the pH of the reaction medium has changed.

28. The method as claimed in claim 20, wherein the amount of functional substance is within the range of from about 0.1 to about 40 moles per mole of pharmaceutical drug.

29. The method as claimed in claim 28, wherein the amount of functional substance is within the range of from about 0.5 to about 5 moles per mole of pharmaceutical drug.

30. A method of treating a cosmetic condition or dermatologic indication in a subject comprising topically administering a therapeutically effective amount of the composition as claimed in claim 1 to a subject in need thereof.

31. The method as claimed in claim 30, wherein the pH of the composition is within the range of from about 2.0 to about 7.0

32. The method as claimed in claim 31, wherein the pH of the composition is within the range of from about 3.0 to about 5.0.

33. The method as claimed in claim 30, wherein the composition is in a form selected from the group consisting of lotion, cream, ointment, and gel.

34. The method as claimed in claim 33, wherein the composition additionally includes a cosmetically or dermatologically acceptable excipient.

35. The method as claimed in claim 30, wherein the method treats, heals or prevents a cosmetic condition or dermatological indication.

36. The method as claimed in claim 35, wherein the cosmetic condition or dermatological indication is selected from the group consisting of: disturbed keratinization; inflammation; defective syntheses of dermal components; changes associated with intrinsic and extrinsic aging of skin, nail and hair; dryness or looseness of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; dermatoses; eczema; psoriasis; pruritus; warts; herpes; age spots; lentigines; melasmas; blemished skin; hyperkeratoses; hyperpigmented or hypopigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubticants and luster; dull and older-looking skin, nail and hair; fragility and splitting of nail and hair, or used as to lighten the skin.
